(19) United States
(12) Patent Application Publication
(10) Pub. No.: US 2017/0239084 A1
(43) Pub. Date: Aug. 24, 2017

(54) SYSTEM FOR LOCAL THERMAL TREATMENT

(71) Applicant: Cevilo, Inc., Andover, MA (US)

(72) Inventor: Henry Calderon, Andover, MA (US)

(21) Appl. No.: 15/587,743

(22) Filed: May 5, 2017

Related U.S. Application Data
(63) Continuation of application No. 15/362,498, filed on Nov. 28, 2016.
Application No. 15/362,498 is a continuation of application No. PCT/US15/46284, filed on Aug. 21, 2015.
(60) Provisional application No. 62/040,536, filed on Aug. 22, 2014.

(51) Int. Cl. A61F 7/02; A61F 7/00

(52) U.S. Cl. CPC A61F 7/02; A61F 7/007; A61F 2007/0093; A61F 2007/0096; A61F 2007/0078; A61F 2007/0219

(57) ABSTRACT

A system for local thermal treatment includes a solid-state heat pump, a controller, a power supply, a heat sink, a thermal conductor, and a thermal pack. The solid-state heat pump may increase or decrease the temperature of the thermal pack to a desired temperature for providing heat or cold treatment. The controller provides control of the solid-state heat pump and its associated components. The heat sink may include an air heat exchanger with fins and a fan, and a liquid heat exchanger with a coolant loop and pump. The coolant loop may allow the heat sink to be separated from the thermal pack for convenient use in constrained spaces. The thermal conductor and thermal pack may be flexible and may be configured specifically to conform to individual body parts. The thermal pack provides local thermal treatment to a subject's body for an extended duration.

### RELATED APPLICATIONS

This application is a continuation of U.S. application Ser. No. 15/362,498, filed on Nov. 28, 2016, which is a continuation of International Application No. PCT/US15/46284, filed on Aug. 21, 2015, which claims priority to U.S. Provisional Application No. 62/040,536, filed Aug. 22, 2014. The entire content of the aforementioned applications is hereby incorporated by reference in its entirety.

### FIELD OF THE INVENTION

The invention relates to the field of solid state heat pumps used for temperature control. Solid state heat pumps consist of two types of semiconductor material, such as a p-type and an n-type, aligned in parallel. When an electrical junction connects one end of the materials, and a voltage is applied across the opposite end, flow of electrical current through the dissimilar materials causes a temperature difference between the two ends. As a result, heat moves from one end of a solid state heat pump to the other. If the applied voltage polarity is reversed, heat flows in the opposite direction. This enables heating and cooling from a single device, which is useful for versatile temperature control.

### BACKGROUND

Self-heating devices that produce heat through exothermic chemical reactions are known to the art, as well as devices for producing heat or cold by heat of dilution rather than by chemical reaction. Typically, these devices are not reusable and the duration for which they can provide heating or cooling is limited.

There is a need in the art for a heating or cooling device that is portable, but also able to provide heating or cooling for an extended time period.

### SUMMARY OF THE INVENTION

A system for local thermal treatment includes a solid-state heat pump, a controller, a power supply, a heat sink, a thermal conductor, and a thermal pack. The thermal pack provides local thermal treatment to a subject's body for an extended duration.

### BRIEF DESCRIPTION OF THE FIGURES

FIG. 1 is a block diagram showing one system for local thermal treatment, in an embodiment.

FIG. 2 is a block diagram showing one system for local thermal treatment, in another embodiment.

### DETAILED DESCRIPTION

The disclosure provides an electrical cooling and heating pack. The electrical cooling pack is capable of cooling body parts for various therapies and treatments. The electrical cooling pack can be portable and/or hand held. It can also be installed into furniture, for example a seat or chair for the application of cold to the body of a subject. It can also be installed in clothing including a hat or helmet.

The disclosure further provides a system for local thermal treatment, wherein the system operates from electrical power in order to provide heating or cooling that can be applied to a part of the body of a subject. The systems described herein can be used to apply thermal treatments for a variety of reasons including muscle therapy, stiffness, pain, strains, sprains or muscle tears. The systems described herein can be used to apply thermal treatments for reducing symptoms associated with insomnia and central nervous system disorders. The systems described herein can also be used for medical and surgical purposes. For example, surgeries where the application of cold using the systems described herein could be used post surgically include abdominal surgeries like Caesarean section, appendectomy, hernia surgery and abdominoplasty (tummy tuck); head and neck surgeries like vocal cord surgery and tumor removal in the oral cavity; orthopedic surgeries like meniscus tear repair surgery, knee surgery, shoulder surgery, hand surgery, hip surgery and foot surgery; as well as cardiac surgeries like coronary stent implantation, cardiac ablation and bypass surgery. The systems described herein can also be used to provide heat in cool environments or cold in hot environments, thereby keeping a subject in a relatively moderate temperature. In certain embodiments, the system for local thermal treatment is portable. In these embodiments, the system can be small enough to be carried by hand or it can be installed into a seat of a vehicle. In other embodiments, the systems described herein are intended to be stationary. In these embodiments, the systems are installed into furniture or other appliances for the application of heat or cold to a subject.

In certain embodiments, the system weighs between 0.5 and 15 pounds. In other embodiments, the system weighs between 0.25 and 10, 1 and 8, 3 and, 4 and 5 or 0.25 and 1 pounds. In certain embodiments, the system has a length of between 2 inches and 4 feet. In other embodiments, the system has a length of between 3 inches and 3 feet or 6 inches and 2 feet. In certain embodiments, the system has a width of 2 inches and 4 feet. In other embodiments, the system has a length of between 3 inches and 3 feet or 6 inches and 2 feet. In certain embodiments, the system has a depth of between 2 inches and 4 feet. In other embodiments, the system has a length of between 3 inches and 3 feet or 6 inches and 2 feet.

In certain embodiments, the system could include two or more thermal treatment devices that are worn on distinct parts of the body or are linked together on a single part of the body. In some embodiments, the system could include 2-20 thermal treatment devices.

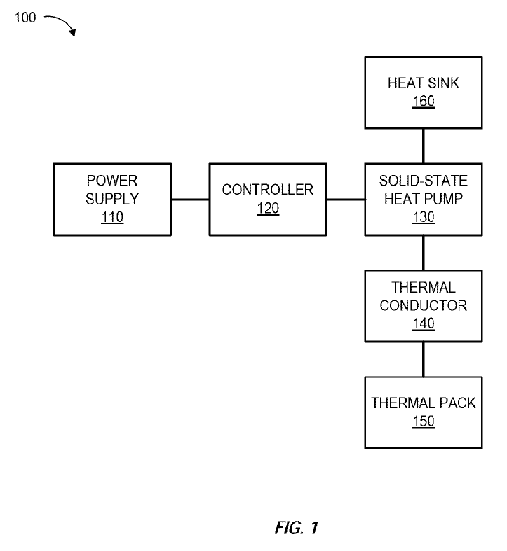

FIG. 1 is a block diagram showing one system 100 for local thermal treatment, in an embodiment. A power supply 110 provides electrical power to system 100. A controller 120 provides control of system 100 components. A solid-state heat pump 130 pumps heat in response to an applied voltage. Solid state heat pump 130 includes a p-type and an n-type semiconductor material aligned in parallel, and an electrical junction connecting the two materials at one end. When a voltage is applied across the two materials at their opposite end, flow of electrical current through the dissimilar materials causes a temperature difference between the two ends. As a result, heat moves from one end of solid state heat pump 130 to the other, forming a hot end and a cold end. A thermal conductor 140 is thermally connected to one end of solid-state heat pump 130, such that heat is transferred between solid-state heat pump 130 and thermal conductor 140 by conduction. Thermal conductor 140 may be made of any material with sufficient thermal conductivity, such as a metal. A thermal pack 150 is thermally connected to thermal conductor 140, such that heat is transferred between thermal conductor 140 and thermal pack 150 by conduction. A heat sink 160 is thermally connected to solid-state heat pump 130 on the opposite end of thermal conductor 140 and thermal pack 150, such that heat is transferred between solid-state heat pump 130 and heat sink 160 by conduction.

Thermal pack 150 may apply thermal treatment to a subject's body. Commonly used hot or cold packs, which do not include a power supply, only remain hot or cold for a limited amount of time. In situations where thermal treatment is desired for longer durations, thermal pack 150 maintains a desired temperature. Another advantage of thermal pack 150 is the option to alternate between heat and cold treatment with the same device.

Controller 120 has electronic circuitry including relays and switches. In an embodiment, controller 120 includes a small digital computer, such as a programmable controller, a programmable logic controller, or a programmable logic relay. Controller 120 includes non-transitory instructions, stored in non-volatile memory, wherein the instructions, when executed by the computer, perform steps for controlling other components of system 100. Control of solid-state heat pump 130 by controller 120 maintains thermal pack 150 at a desired temperature. In an embodiment, the desired temperature may be any temperature within a desired range. For example, voltage applied to solid-state heat pump 130, under control of controller 120, may cool thermal conductor 140 and thermal pack 150 to a temperature between +4° C. and −20° C. for cold treatment. Alternatively, the voltage polarity may be reversed by controller 120 to heat thermal conductor 140 and thermal pack 150 to a temperature between 40° C. and 50° C. for heat treatment. In an embodiment, temperature control includes applying a voltage to solid-state heat pump 130 for a pre-determined amount of time to achieve a desired temperature, after which, a desired temperature range is maintained by lowering the voltage applied to solid-state heat pump 130 or by cycling the voltage on and off

A temperature difference across solid-state heat pump 130 is determined by its properties, such as its size, the materials used, and how it was constructed. Solid-state heat pump 130 is appropriately selected and sized to achieve sufficiently high and low temperatures in thermal pack 150. Solid-state heat pump 130 is also appropriately selected and sized to enable rapid temperature change in thermal pack 150. The achievable high and low temperatures of solid-state heat pump 130 depend on the ambient air temperature and the ability of heat sink 160 to add or remove heat. In the case where thermal pack 150 is cooled, thermal conductor 140 and solid-state heat pump 130 operate in conjunction to pull heat from thermal pack 150, thereby lowering the temperature thereof. Any excess heat pulled from thermal pack 150 is then discharged into the surrounding medium via a thermal dissipator like a heat sink 160 thereby increasing the efficiency of the heat pump 130. Conversely, when thermal pack 150 is heated, solid-state heat pump 130 and thermal conductor 140 operate in conjunction to heat thermal pack 150. Heat sink 160 operates to discharge any coolness from solid-state heat pump 130 into the surrounding medium, thereby increasing the efficiency of solid-state heat pump 130.

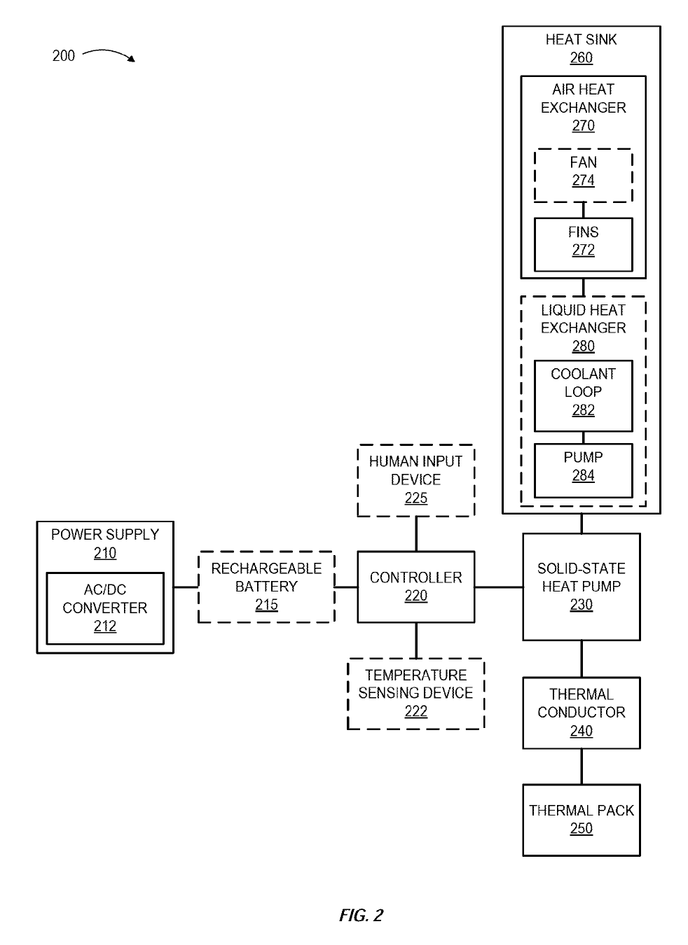

FIG. 2 is a block diagram showing one system 200 for local thermal treatment. A power supply 210, which provides electrical power to system 200, includes an AC/DC converter 212 that converts electricity from alternating current to direct current. Power supply 210 for example converts “wall power” into energy that is usable by system 200. In one embodiment, an optional rechargeable battery 215 provides power to system 200 and is recharged by power supply 210. Rechargeable battery 215 improves the portability of system 200 and allows it to be used remotely from power supply 210. Alternately, or in addition to rechargeable battery 215, controller 220 may connect to power supply 210 via a cord such that system 200 operates when power supply 210 is coupled to an outlet. A controller 220 provides control of electrical power to the components of system 200. Controller 220 has electronic circuitry including relays and switches. In an embodiment, controller 220 includes a small digital computer, such as a programmable controller, a programmable logic controller, or a programmable logic relay. Controller 220 includes non-transitory instructions, stored in non-volatile memory, wherein the instructions, when executed by the computer, perform steps for controlling other components of system 200. An optional human input device 225 connects to controller 220 enabling a user to input information. Human input device 225 may include, without being limited to, the following examples: one or more switches, a dial, or a graphic user interface (GUI) manipulated by buttons, a keyboard, a mouse, a touchscreen, a phone or a watch. The GUI may be used remotely from the rest of the system. In certain embodiments, the GUI communicates with the controller via Bluetooth or any other wireless electronic method. A switch may be used to select between on and off, or between hot and cold. A dial may be used to select a temperature range or set point. A GUI may be used to select, via the buttons, a temperature set point or a profile of temperature set points. The GUI may also be used to set a timer for maintaining temperature over a desired interval, or to set a clock for changing temperature at a desired time. An optional temperature sensing device 222 may be electrically connected to provide temperature information to controller 220. For example, temperature sensing device may be configured to measure temperature inside, and near the surface of, thermal pack 150. Examples of temperature sensing device 222 include, but are not limited to, a thermocouple or a resistance temperature detector.

A solid-state heat pump 230 pumps heat in response to an applied voltage. Solid state heat pump 230 includes a p-type and an n-type semiconductor material aligned in parallel, and an electrical junction connecting the two materials at one end. When a voltage is applied across the two materials at their opposite end, flow of electrical current through the dissimilar materials causes a temperature difference between the two ends. As a result, heat moves from one end of solid state heat pump 230 to the other, forming a hot end and a cold end. A thermal conductor 240 is thermally connected to one end of solid-state heat pump 230, thereby transferring heat between thermal conductor 240 and solid-state heat pump 230 by conduction. Thermal conductor 240 may be made of any material with sufficient thermal conductivity, such as a metal. In an embodiment, thermal conductor 240 is made of a flexible material. A thermal pack 250 is thermally connected to thermal conductor 240, such that heat is transferred between thermal pack 250 and thermal conductor 240 by conduction. In an embodiment, thermal pack 250 includes a gel encased in a flexible package, wherein thermal pack 250 remains flexible when cold. In an alternate embodiment, thermal pack 250 includes a plurality of beads encased in a flexible package, wherein thermal pack 250 remains flexible when cold. A heat sink 260 is thermally connected to solid-state heat pump 230 on the opposite end of thermal conductor 240 and thermal pack 250. Thermal pack 250 is used to apply thermal treatment to a subject's body. In situations where thermal treatment is desired for long durations, and power supply 210 is unavailable, rechargeable battery 215 provides power for thermal pack 250 to maintain a desired temperature. Thermal pack 250, including flexible thermal conductor 240, may be sized and shaped for specific thermal treatments. This may include, but is not limited to, applying thermal treatment to an ankle, knee, elbow, wrist, finger, shoulder, lower back, upper back, neck, head, or any body part or group of body parts.

Heat sink 260 is thermally connected to solid-state heat pump 230 at the end opposite thermal conductor 240 and thermal pack 250. If thermal pack 250 is cooled, heat sink 260 discharges excess heat into the surrounding medium. If the polarity of the voltage is reversed to heat thermal pack 250, heat sink 260 discharges excess coolness into the surrounding medium. Heat sink 260 includes an air heat exchanger 270, which exchanges heat with the ambient air. Air heat exchanger 270 is made of a material with sufficient thermal conductivity, such as a metal. In an embodiment, air heat exchanger 270 is made of anodized aluminum due to its sufficient thermal conductivity, light weight, and durability. Air heat exchanger 270 includes fins 272, which provide an increased surface area for a given volume. This increased surface area increases the rate of heat exchange with the air. An optional fan 274 blows air across the fins, thereby further increasing the rate of heat exchange. Controller 220 controls the speed of fan 274, or turns it on or off as needed.

In addition to air heat exchanger 270, heat sink 250 may include an optional liquid heat exchanger 280. Liquid heat exchanger 280 provides increased heat transfer due to the higher density, and thus larger heat carrying capacity, of liquids compared to air. Another advantage afforded by liquid heat exchanger 280 is the option to physically distance heat sink 260 from thermal pack 250 with a sufficiently long coolant loop 282. Separation from heat sink 260 allows thermal pack 250 to be used in a constrained space, such as between a subject and a seat or bed, while maintaining sufficient air exposure to heat sink 260. Liquid heat exchanger 280 is thermally connected to air heat exchanger 270 and one end of solid-state heat pump 230. Liquid heat exchanger 280 includes a coolant loop 282 and a pump 284. Coolant loop 282 forms a continuous loop that recycles coolant between thermal contact points of air heat exchanger 270 and solid-state heat pump 230, thereby transferring heat between them. Controller 220 controls the flow rate of pump 284, or turns it on or off as needed. Pump 284 may be any pump suitable for driving flow of liquid within coolant loop 282. In an embodiment, pump 284 is a peristaltic pump, which drives flow by squeezing the coolant loop tubing and therefore does not contact the coolant liquid. Coolant loop 282 contains a fluid that remains in a liquid state at both temperature extremes of solid-state heat pump 230. In an embodiment, the coolant liquid is a mixture of water and propylene glycol. Coolant loop 284 may be constructed of tubes made of any material compatible with the coolant liquid, pump 284, and the high and low temperature extremes produced by solid-state heat pump 230. In other words, the tube material must substantially prevent penetration and corrosion by the coolant liquid, and it must be sufficiently flexible for squeezing by a peristaltic pump, at both temperature extremes. In an embodiment, coolant loop 282 consists of platinum-cured silicon tubing.

In embodiments where the system is installed in a seat or bed, the seat or bed can be installed in a vehicle. In some embodiments, the vehicle is a car, truck, boat, bus, train, airplane or helicopter. The seat or bed can be used by the driver or pilot or by a passenger. In other embodiments, the seat or bed can be furniture that is used in the home or office.

In an embodiment, controller 220 includes one or more relays for changing voltage polarity and one or more switches for applying voltage. In an embodiment, controller 220 includes an algorithm that controls voltage supplied to solid-state heat pump 230 using the one or more relays and one or more switches. Controller 220 identifies a voltage differential measurement indicating any difference between a desired temperature and a measured temperature from temperature sensing device 222. The desired temperature may be predetermined or entered by a user via human input device 225. Based on the temperature difference, controller 220 sends a control signal to solid-state heat pump 230 to adjust the voltage to solid-state heat pump 230 according to the control algorithm, thereby bringing the measured temperature closer to the desired temperature. Controller 220 may also send control signals to adjust power supplied to fan 274 and pump 284 to appropriately transfer heat. Parameters of the control algorithm are tuned to achieve a desired response. Optionally, the control algorithm parameters may be adjusted using human input device 225.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A system for local thermal treatment, comprising:
at least one solid-state heat pump;
a controller to control the at least one solid-state heat pump;
a power supply to provide energy to the solid-state heat pump under control of the controller;
a heat sink to discharge heat from the system;
a thermal pack for applying local thermal treatment to a body for an extended duration; and
a thermal conductor coupled to the at least one solid-state heat pump and the thermal pack providing a heat transfer path therebetween.

2. The system of claim 1, the heat sink comprising:
an air heat exchanger thermally coupled to the at least one solid-state heat pump to dissipate heat into the air.

3. The air heat exchanger of claim 2, comprising:
a plurality of fins thermally connected to the at least one solid-state heat pump; and
a fan oriented to move air across the fins thereby aiding removal of heat from the fins.

4. The system of claim 1, the heat sink comprising:
a liquid heat exchanger including:
a coolant loop thermally coupled between an air heat exchanger and the at least one solid-state heat pump; and
a pump for moving a liquid within the coolant loop thereby transferring heat between the air heat exchanger and the solid-state heat pump.

5. The system of claim 1, the thermal pack comprising:
a gel encased in a flexible package, the gel maintaining thermal pack flexibility when cold.

6. The system of claim 1, the thermal pack comprising:
a plurality of beads encased in a flexible package, wherein the package remains flexible when cold.

7. The system of claim 1, the power supply comprising:
an AC/DC power converter.

8. The system of claim 1, further comprising:
a rechargeable battery, wherein the rechargeable battery provides power to the system,
wherein the power supply is removably coupled to the rechargeable battery to recharge the rechargeable battery.

9. The system of claim 1, the controller comprising:
one or more relays and one or more switches; and
a programmable controller configured to control the one or more relays and the one or more switches to control a voltage supplied to the solid-state heat pump.

10. The system of claim 1, comprising:
a sensing device to measure temperature in the thermal pack; and
a human input device for setting a temperature set point of the thermal pack.

11. The system of claim 9, the controller comprising:
a control algorithm for controlling a voltage supplied to the solid-state heat pump based upon a temperature difference between the temperature measured by the sensing device and the temperature set point.

12. The system of claim 9, the controller comprising:
a control algorithm for controlling a voltage supplied to the fan of the air heat exchanger, thereby controlling the fan speed and for controlling a voltage supplied to the pump of the liquid heat exchanger, thereby controlling the pump flow rate.

13. A method of applying a thermal treatment to a subject in need thereof comprising applying to the subject a system for local thermal treatment, comprising:
at least one solid-state heat pump;
a controller to control the at least one solid-state heat pump;
a power supply to provide energy to the solid-state heat pump under control of the controller;
a heat sink to discharge heat from the system;
a thermal pack for applying local thermal treatment to a body for an extended duration; and
a thermal conductor coupled to the at least one solid-state heat pump and the thermal pack providing a heat transfer path therebetween.

14. The method of claim 13, wherein the system is applied to at least one muscle of the subject.

15. The method of claim 14, wherein the subject suffers from stiffness, pain, strains, sprains or muscle tears.

16. The method of claim 13, wherein the system is applied to treat post-surgical symptoms.

17. The method of claim 16, wherein the surgery is selected from the group consisting of Caesarean section, appendectomy, hernia surgery, abdominoplasty (tummy tuck); head and neck surgeries; orthopedic surgeries and cardiac surgeries.
